(19) United States
(12) Patent Application Publication
(10) Pub. No.: US 2024/0105344 A1
(43) Pub. Date: Mar. 28, 2024

(54) SYSTEM FOR CONSTRUCTING HYPERKALEMIA PREDICTION ALGORITHM THROUGH ELECTROCARDIOGRAM, METHOD FOR CONSTRUCTING HYPERKALEMIA PREDICTION ALGORITHM THROUGH ELECTROCARDIOGRAM BY USING SAME, AND HYPERKALEMIA PREDICTION SYSTEM USING ELECTROCARDIOGRAM

(71) Applicant: UNIVERSITY INDUSTRY FOUNDATION, YONSEI UNIVERSITY WONJU CAMPUS, Wonju-si, Gangwon-do (KR)

(72) Inventors: Hyun YOUK, Wonju-si (KR); Sang Won HWANG, Seoul (KR); ERDENEBAYAR URTNASAN, Wonju-si (KR)

(73) Assignee: UNIVERSITY INDUSTRY FOUNDATION, YONSEI UNIVERSITY WONJU CAMPUS, Wonju-si, Gangwon-do (KR)

(21) Appl. No.: 18/527,458

(22) Filed: Dec. 4, 2023

Related U.S. Application Data
(63) Continuation of application No. PCT/KR2022/008022, filed on Jun. 7, 2022.

(30) Foreign Application Priority Data
Jun. 4, 2021 (KR) 10-2021-0073050
Jun. 4, 2021 (KR) 10-2021-0073051

(51) Int. Cl. G16H 50/50; A61B 5/00; A61B 5/28; G16H 50/20; G16H 50/70

(52) U.S. Cl. CPC G16H 50/50; A61B 5/28; A61B 5/681; G16H 50/20; G16H 50/70

(57) ABSTRACT

The present invention relates to a system for constructing a hyperkalemia prediction algorithm through an electrocardiogram, a method for constructing the hyperkalemia prediction algorithm through the electrocardiogram by using the same, and a hyperkalemia prediction system using the electrocardiogram, and the system for constructing a hyperkalemia prediction algorithm through an electrocardiogram includes: a data collection unit collecting electrocardiogram data of multiple hyperkalemia patients; a data processing unit generating a training dataset for machine learning based on the electrocardiogram data collected by the data collection unit; and a model generation unit constructing a neural network model for predicting a hyperkalemia using an electrocardiogram based on the training dataset provided by the data processing unit.

### CROSS-REFERENCE TO RELATED APPLICATIONS

This application claims the priority of Korean Patent Application No. 10-2021-0073050 and No. 10-2021-0073051, both filed on Jun. 4, 2021, with the Korean Intellectual Property Office, the disclosure of which is incorporated herein by reference.

### BACKGROUND OF THE INVENTION

### Field of the Invention

The present invention relates to a system for constructing a hyperkalemia prediction algorithm through an electrocardiogram, a method for constructing the hyperkalemia prediction algorithm through the electrocardiogram by using the same, and a hyperkalemia prediction system using the electrocardiogram, and more particularly, to a system which can construct a neural network model capable of predicting a hyperkalemia using an electrocardiogram, a method for constructing the hyperkalemia prediction algorithm through the electrocardiogram by using the same, and a hyperkalemia prediction system using the electrocardiogram.

### Description of the Related Art

Hyperkalemia is an electrolyte displacement that can lead to fatal cardiac arrhythmias. Proper management of hyperkalemia is becoming more important due to the increasing incidence of hyperkalemia-related diseases such as diabetes, coronary artery disease, and chronic kidney disease. Hyperkalemia and hypokalemia, or fluctuations in potassium levels, are both associated with an increased risk of death and life-threatening arrhythmias. In patients with kidney disease or heart disease, morbidity, hospitalization, and mortality can follow modest changes in potassium levels.

Meanwhile, to measure potassium concentration in the body, an invasive method called blood sampling is used. Therefore, patients suffering from potassium-related diseases such as hyperkalemia must visit the hospital and get tested to measure potassium concentration. However, the concentration of potassium in the body, which can have fatal effects, requires constant management, but measuring it through a hospital visit every time is inconvenient for both the hospital and the patient, making it difficult to manage.

### SUMMARY OF THE INVENTION

The present invention is contrived to improve such a problem, and has been made in an effort to provide a system which can construct an algorithm capable of predicting a hyperkalemia using an electrocardiogram, a method for constructing the hyperkalemia prediction algorithm through the electrocardiogram by using the same, and a hyperkalemia prediction system using the electrocardiogram.

In order to achieve the object, an exemplary embodiment of the present invention provides a system for constructing a hyperkalemia prediction algorithm through an electrocardiogram, including: a data collection unit collecting electrocardiogram data of multiple hyperkalemia patients; a data processing unit generating a training dataset for machine learning based on the electrocardiogram data collected by the data collection unit; and a model generation unit constructing a neural network model for predicting a hyperkalemia using an electrocardiogram based on the training dataset provided by the data processing unit.

The data collection unit collects electrocardiogram data of patients who have developed symptoms of hyperkalemia.

The data collection unit may collect electrocardiogram data of the hyperkalemia patient data from a time before a first preset reference time from the time of the symptom onset of the hyperkalemia to a time after a second preset reference time from the time of the symptom onset of the hyperkalemia.

The data processing unit classifies the ECG data at the time of the symptom onset of the hyperkalemia among the ECG data provided by the data collection unit into abnormal-state data, and classifies the ECG data from the time of the symptom onset of the hyperkalemia to a time before a first preset reference time into normal-state data, and generates the training dataset to include the normal-state data and the abnormal-state data which are classified.

The data processing unit classifies the ECG data at the time of the symptom onset of the hyperkalemia among the ECG data provided by the data collection unit into abnormal-state data, and classifies the ECG data from the time of the symptom onset of the hyperkalemia to a time after a second preset reference time into normal-state data, and generates the training dataset to include the normal-state data and the abnormal-state data which are classified.

The data processing unit may classify the ECG data at the time of the symptom onset of the hyperkalemia among the ECG data provided by the data collection unit into the abnormal-state data, and also classify, into the normal-state data, ECG data other than the ECG data among the ECG data from the time before the first preset reference time from the time of the symptom onset of the hyperkalemia to the time after the second preset reference time from the time of the symptom onset of the hyperkalemia, and generate the training dataset to include the normal-state data and the abnormal-state data which are classified.

The data processing unit may classify some randomly selected data of the data classified from the ECG data provided by the data collection unit into the training dataset, and classify the remaining data into a sample set for testing or validating the neural network model.

The ECG data collected by the data collection unit is preferably ECG lead II signals data.

The symptom of the hyperkalemia is chronic renal failure (CRF).

Meanwhile, the system for constructing the hyperkalemia prediction algorithm through an electrocardiogram may further include a validation module analyzing the performance of the neural network model by applying the sample set to the neural network model.

Meanwhile, another exemplary embodiment of the present invention provides a method for constructing a hyperkalemia prediction algorithm through an electrocardiogram, including: data collection step of colleting, by a data collection unit, electrocardiogram data of multiple hyperkalemia patients; a data processing step of generating, by a data processing unit, a training dataset for machine learning based on the electrocardiogram data collected in the data collection step; and a model generation step of constructing, by a model generation unit, a neural network model for predicting a hyperkalemia using an electrocardiogram based on the training dataset.

In the data collection step, electrocardiogram data of patients who have developed symptoms of hyperkalemia are collected.

In the data collection step, electrocardiogram data of the hyperkalemia patient data from a time before a first preset reference time from the time of the symptom onset of the hyperkalemia to a time after a second preset reference time from the time of the symptom onset of the hyperkalemia may be collected.

In the data processing step, the ECG data at the time of the symptom onset of the hyperkalemia among the ECG data collected in the data collection step may be classified into abnormal-state data, and the ECG data from the time of the symptom onset of the hyperkalemia to a time before a first preset reference time may be classified into normal-state data, and the training dataset may be generated to include the normal-state data and the abnormal-state data which are classified.

In the data processing step, the ECG data at the time of the symptom onset of the hyperkalemia among the ECG data collected in the data collection step may be classified into abnormal-state data, and the ECG data from the time of the symptom onset of the hyperkalemia to a time after a second preset reference time may be classified into normal-state data, and the training dataset may be generated to include the normal-state data and the abnormal-state data which are classified.

In the data processing step, the ECG data at the time of the symptom onset of the hyperkalemia among the ECG data collected in the data collection step may be classified into the abnormal-state data, and ECG data other than the ECG data among the ECG data from the time before the first preset reference time from the time of the symptom onset of the hyperkalemia to the time after the second preset reference time from the time of the symptom onset of the hyperkalemia may be classified into the normal-state data, and the training dataset may be generated to include the normal-state data and the abnormal-state data which are classified.

In the data processing step, some randomly selected data of the data classified from the ECG data collected in the data collection step may be classified into the training dataset, and the remaining data may be classified into a sample set for testing or validating the neural network model.

Meanwhile, the method for constructing the hyperkalemia prediction algorithm through an electrocardiogram may further include a validation step of analyzing the performance of the neural network model by applying the sample set to the neural network model.

Meanwhile, yet another exemplary embodiment of the present invention provides a hyperkalemia prediction system using an electrocardiogram, including: a smart band worn by a user, and measuring an electrocardiogram of the user; an information collection unit collecting information on the electrocardiogram of the user measured by the smart band; and a determination module determining whether the user has thee hyperkalemia by applying the electrocardiogram of the user collected by the information collection unit to a neural network model pre-constructed to predict the hyperkalemia according to the electrocardiogram.

The smart band is preferably worn on a wrist of the user.

Meanwhile, the hyperkalemia prediction system using an electrocardiogram may further include a model construction unit constructing the neural network mode using electrocardiogram data of the hyperkalemia patient, and providing the constructed neural network model to the determination module.

The model construction unit includes a data collection unit collecting electrocardiogram data of multiple hyperkalemia patients; a data processing unit generating a training dataset for machine learning based on the electrocardiogram data collected by the data processing unit; and a model generation unit constructing a neural network model for predicting a hyperkalemia using an electrocardiogram based on the training dataset provided by the data processing unit.

The data collection unit may collect electrocardiogram data of the hyperkalemia patient data from a time before a first preset reference time from the time of the symptom onset of the hyperkalemia to a time after a second preset reference time from the time of the symptom onset of the hyperkalemia.

The data processing unit may classify the ECG data at the time of the symptom onset of the hyperkalemia among the ECG data provided by the data collection unit into abnormal-state data, and classify the ECG data from the time of the symptom onset of the hyperkalemia to the time before the first preset reference time into normal-state data, and generate the training dataset to include the normal-state data and the abnormal-state data which are classified.

The data processing unit may classify the ECG data at the time of the symptom onset of the hyperkalemia among the ECG data provided by the data collection unit into abnormal-state data, and classify the ECG data from the time of the symptom onset of the hyperkalemia to the time after the second preset reference time into normal-state data, and generate the training dataset to include the normal-state data and the abnormal-state data which are classified.

The data processing unit may classify the ECG data at the time of the symptom onset of the hyperkalemia among the ECG data provided by the data collection unit into the abnormal-state data, and classify, into the normal-state data, ECG data other than the ECG data among the ECG data from the time before the first preset reference time from the time of the symptom onset of the hyperkalemia to the time after the second preset reference time from the time of the symptom onset of the hyperkalemia, and generate the training dataset to include the normal-state data and the abnormal-state data which are classified.

The data processing unit may classify some randomly selected data of the data classified from the ECG data provided by the data collection unit into the training dataset, and classify the remaining data into a sample set for testing or validating the neural network model.

Meanwhile, the hyperkalemia prediction system using an electrocardiogram may further include a validation module analyzing the performance of the neural network model by applying the sample set to the neural network model.

According to the present invention, in a system for constructing a hyperkalemia prediction algorithm through an electrocardiogram, a method for constructing the hyperkalemia prediction algorithm through the electrocardiogram by using the same, and a hyperkalemia prediction system using the electrocardiogram, since a neural network model can be constructed for determining whether a patient has the hyperkalemia using the electrocardiogram, there is an advantage in being able to more easily diagnose patients with the hyperkalemia in a non-invasive manner using the neural network model.

### BRIEF DESCRIPTION OF THE DRAWINGS

FIG. 1 is a block diagram of a system for constructing a hyperkalemia prediction algorithm through an electrocardiogram according to the present invention;

FIG. 2 is a flowchart for a method for constructing a hyperkalemia prediction algorithm through an electrocardiogram according to the present invention;

FIG. 3 is a conceptual view of a hyperkalemia prediction system using an electrocardiogram according to the present invention; and

FIG. 4 is a block diagram of the hyperkalemia prediction system using an electrocardiogram according to the present invention.

### DETAILED DESCRIPTION OF THE PREFERRED EMBODIMENT

Hereinafter, a system for constructing a hyperkalemia prediction algorithm through an electrocardiogram, a method for constructing the hyperkalemia prediction algorithm through the electrocardiogram by using the same, and a method for constructing the hyperkalemia prediction algorithm through the electrocardiogram by using the same, and a hyperkalemia prediction system using the electrocardiogram, may have various modifications and various embodiments with reference to the accompanying drawings, so specific exemplary embodiments will be illustrated in the drawings and described in detail in the specification. However, this does not limit the present invention to specific exemplary embodiments, and it should be understood that the present invention covers all the modifications, equivalents and replacements included within the idea and technical scope of the present invention. In describing each drawing, reference numerals refer to like elements. In the accompanying drawings, the sizes of structures are illustrated while being enlarged as compared with actual sizes for clarity of the present invention.

Terms including as first, second, and the like are used for describing various components, but the components should not be limited by the terms. The terms are used only to discriminate one component from another component. For example, a first component may be referred to as a second component, and similarly, the second component may be referred to as the first component without departing from the scope of the present invention.

Terms used in the present application are used only to describe specific exemplary embodiments, and are not intended to limit the present invention. A singular form includes a plural form if there is no clearly opposite meaning in the context. In the present application, it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

If not contrarily defined, all terms used herein including technological or scientific terms have the same meanings as those generally understood by a person with ordinary skill in the art. Terms which are defined in a generally used dictionary should be interpreted to have the same meaning as the meaning in the context of the related art, and are not interpreted as an ideal meaning or excessively formal meanings unless clearly defined in the present application.

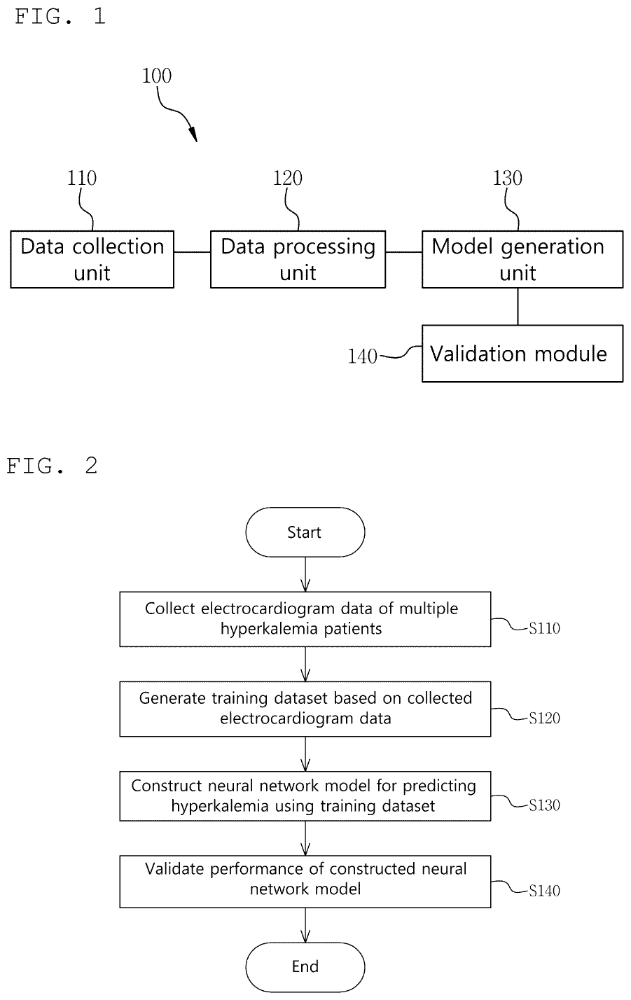

FIG. 1 illustrates a hyperkalemia prediction algorithm constructing system 100 according to the present invention.

Referring to the drawing, the hyperkalemia prediction algorithm constructing system 100 includes a data collection unit 110 collecting electrocardiogram data of multiple hyperkalemia patients, a data processing unit 120 generating a training dataset for machine learning based on the electrocardiogram data collected by the data processing unit 120, and a model generation unit 130 constructing a neural network model for predicting a hyperkalemia using an electrocardiogram based on the training dataset provided by the data processing unit 120.

The data collection unit 110 collects electrocardiogram data of multiple patients measured through an electrocardiogram measurement device (not illustrated). In respect to the electrocardiogram data, a plurality of electrocardiogram data may be measured through multiple measurement channels depending on the number of electrodes attached to the patient's body. Here, the electrocardiogram measurement device adopts a device that measures 12-lead electrocardiography (ECG) using electrodes of lead I, lead II, lead III, aVR, aVL, aVF, V1, V2, V3, V4, V5, and V6, and the data collection unit 110 may collect ECG data input from each electrode of the ECG measurement device. Here, the data collection unit 110 may be connected to the corresponding ECG measurement device and receive the corresponding ECG data. All 12-lead electrocardiogram data are matched with electrolyte tests within 2 hours of each section to form a dataset. Then, the data collection unit 110 extracts and stores the waveform of the 12-lead ECG signal with a sampling frequency of 700 Hz. Finally, an ECG signal segment is a duration of 2 seconds and consists of 1400 samples.

At this time, the patient who is a hyperkalemic patient whose plasma potassium concentration is higher than the normal level (3.7 to −5.3 mEq/L) has developed symptoms of hyperkalemia. Here, symptoms of hyperkalemia include chronic renal failure, arrhythmia, etc.

Meanwhile, it is desirable that the data collection unit 110 collects electrocardiogram data of the hyperkalemia patient data from a time before a first preset reference time from the time of the symptom onset of the hyperkalemia to a time after a second preset reference time from the time of the symptom onset of the hyperkalemia. Here, the first and second reference times may be set in various ways depending on the patient's condition and a use place of the neural network model to be constructed.

The data processing unit 120 classifies the ECG data collected by the data collection unit 110 into abnormal-state data or normal-state data to generate the training dataset.

Here, the data processing unit 120 classifies the ECG data at the time of the symptom onset of the hyperkalemia among the ECG data provided by the data collection unit 110 into abnormal-state data. Further, the data processing unit 120 classifies the ECG data from the time of the symptom onset of the hyperkalemia to the time before the first reference time among the ECG data provided by the data collection unit 110 into normal-state data.

Meanwhile, the data processing unit 120 is not limited thereto, but may also classify the ECG data to the time after the second reference time from the time of the symptom onset of the hyperkalemia among the ECG data provided by the data collection unit 110 into the normal-state data. In this case, the data processing unit 120 classifies the ECG data at the time of the symptom onset of the hyperkalemia among the ECG data provided by the data collection unit 110 into the abnormal-state data.

Meanwhile, the data processing unit 120 may also classify, into the normal-state data, ECG data other than the ECG data among the ECG data from the time before the first reference time from the time of the symptom onset of the hyperkalemia to the time after the second reference time from the time of the symptom onset of the hyperkalemia in the ECG data provided by the data collection unit 110. In this case, the data processing unit 120 classifies the ECG data at the time of the symptom onset of the hyperkalemia among the ECG data provided by the data collection unit 110 into the abnormal-state data.

Next, the data processing unit 120 classifies some randomly selected data of the normal-state data and the abnormal-state data classified from the ECG data provided by the data collection unit 110 into the training dataset, and classifies the remaining data into a sample set for testing or validating the neural network model. Here, the sample set is classified into a validation set for validating the performance of the neural network model and a test set for testing the neural network model. At this time, it is desirable that a ratio of the training dataset, the validation set, and the test adopts 6:2:2.

The model generation unit 130 constructs a neural network model for predicting the hyperkalemia using the electrocardiogram based on the training dataset provided by the data processing unit 120.

Here, the neural network model adopts a convolutional neural network (CNN) model, and the CNN model is a hierarchical model used for finally extracting a feature of input data by alternatively performing a plurality of convolutional layers (pooling layers). The neural network model is pre-constructed by processing the training dataset provided by the data processing unit 120 according to a supervised learning technique.

That is, the model generation unit 130 generates the corresponding neural network model as a 5-layer convolutional neural network using one-dimensional convolution operation, max pooling, and fully connected layers. A detailed structure of the corresponding neural network model, that is, a deep learning model, is shown in Table 1 below.

TABLE 1

| | | Acti- | | Output | |
|---|---|---|---|---|---|
| No | Layers | vation | Filter size | shape | Parameter |
| 1 | batchnorm_1 | = | = | 1400 × 1 | 4 |
| 2 | conv1D_1 | relu | 100@50 × 1 | 1351 × 100 | 5,100 |
| | maxpool_1 | | 2 × 1 | 675 × 100 | |
| 3 | conv1D_2 | relu | 80@50 × 1 | 626 × 80 | 400,080 |
| | maxpool_2 | | 2 × 1 | 313 × 80 | |
| | dropout_2 | | p = 0.25 | 313 × 80 | |
| 4 | conv1D_3 | relu | 60@30 × 1 | 284 × 60 | 144,060 |
| | maxpool_3 | | 2 × 1 | 142 × 60 | |
| | dropout_3 | | p = 0.25 | 142 × 60 | |
| 5 | conv1D_4 | relu | 40@20 × 1 | 123 × 40 | 48,040 |
| | maxpool_4 | | 2 × 1 | 61 × 40 | |
| | dropout_4 | | p = 0.25 | 61 × 40 | |
| 6 | conv1D_5 | relu | 20@10 × 1 | 52 × 20 | 8,020 |
| | maxpool_5 | | 2 × 1 | 26 × 20 | |
| | dropout_5 | | p = 0.25 | 26 × 20 | |
| 7 | flattern_1 | softmax | 2 | 520 × 20 | 1,042 |
| | dense_1 | | | | |
| Total | 5 conv. | | 124 filters | | 606,027 |
| | layers | | | | |

Meanwhile, the neural network model is not limited thereto, and any neural network model that may predict the hyperkalemia using the patient's electrocardiogram data may be applied.

Here, the model generation unit 130 may generate the neural network model for each electrode from which ECG data included in each training dataset is obtained.

Meanwhile, the system 100 for constructing the hyperkalemia prediction algorithm through the electrocardiogram according to the present invention may further include a validation module 140 calculating the accuracy of the neural network model by applying the sample set to the neural network model. The validation module 140 calculates the precision and recall of the corresponding neural network model, and calculates performance based on the calculated precision and recall.

Here, the validation module 140 calculates the precision and recall of the neural network model constructed by the model generation unit 130 using Equation 1 below.

$$precision = \frac{T P}{T P + F P} \qquad [ Equation 1 ]$$

$$recall = \frac{T P}{T P + F N}$$

Here, precision represents the precision of the neural network model, recall represents the recall of the neural network model, TP represents a true positive, FP represents a false positive, and FN represents a false negative.

At this time, the validation module 140 calculates the corresponding precision and recall using the normal-state data or abnormal-state data of the validation set and the dataset, respectively. Here, when the validation module 140 applies abnormal-state data to the neural network model, TP which is the true positive adopts the number of times the neural network model predicts actual abnormal-state data as a hyperkalemia state, FP which is the false positive adopts the number of times the neural network model predicts the actual normal-state data as the hyperkalemia state, and FN which is the false negative adopts the number of times the neural network model predicts actual abnormal-state data as a normal state.

Here, when the validation module 140 applies the normal-state data to the neural network model, TP which is the true positive adopts the number of times the neural network model predicts the actual normal-state data as the normal state, FP which is the false positive adopts the number of times the neural network model predicts the actual abnormal-state data as the normal state, and FN which is the false negative adopts the number of times the neural network model predicts the actual normal-state data as the hyperkalemia state.

Next, the validation module 140 calculates the performance of the neural network model by applying the calculated precision and recall of the neural network model to Equation 2 below.

$$F 1 = 2 \times \frac{precision \times recall}{precision + recall} \qquad [ Equation 2 ]$$

Here, F1 represents a value for the performance of the neural network model, precision represents the precision of the neural network model, and recall represents the recall of the neural network model. At this time, the validation module 140 may also calculate the performance of the neural network model for each electrode from which ECG data included in the training dataset is obtained. The validation module 140 provides information on F1 calculated by Equation 2, that is, information on the performance of the neural network model to the manager. The manager may supplement the neural network model based on the information on the performance of the neural network model provided by the validation module 140.

Meanwhile, in FIG. 2, a method for constructing a hyperkalemia prediction algorithm through an electrocardiogram according to the present invention is disclosed.

Referring to the drawing, the hyperkalemia prediction algorithm constructing method includes a data collection step S110, a data processing step S120, a model generation step S130, and a validation step S140.

The data collection step S110 is a step in which the data collection unit 110 collects electrocardiogram data from multiple hyperkalemia patients. Here, the data collection unit 110 collects the ECG data from an ECG measurement device installed on the patient. At this time, the patient who is a hyperkalemic patient whose plasma potassium concentration is higher than the normal level (3.7 to −5.3 mEq/L) has developed symptoms of hyperkalemia.

Here, it is desirable that the data collection unit 110 collects electrocardiogram data of the hyperkalemia patient data from a time before a first preset reference time from the time of the symptom onset of the hyperkalemia to a time after a second preset reference time from the time of the symptom onset of the hyperkalemia.

The data processing step S120 is a step of generating a training dataset for machine learning based on the ECG data collected in the data collection step S110. Here, the data processing unit 120 classifies the ECG data collected by the data collection unit 110 into abnormal-state data or normal-state data to generate the training dataset.

At this time, the data processing unit 120 classifies the ECG data at the time of the symptom onset of the hyperkalemia among the ECG data provided by the data collection unit 110 into the abnormal-state data, and classifies the ECG data from the time of the symptom onset of the hyperkalemia to the time before the first reference time into the normal-state data.

Further, the data processing unit 120 classifies the ECG data at the time of the symptom onset of the hyperkalemia among the ECG data provided by the data collection unit 110 into the abnormal-state data, and classifies the ECG data from the time of the symptom onset of the hyperkalemia to the time after the second reference time into the normal-state data.

In addition, Further, the data processing unit 120 may classify the ECG data at the time of the symptom onset of the hyperkalemia among the ECG data provided by the data collection unit 110 into the abnormal-state data, and also classify, into the normal-state data, ECG data other than the ECG data among the ECG data from the time before the first reference time from the time of the symptom onset of the hyperkalemia to the time after the second reference time from the time of the symptom onset of the hyperkalemia.

Next, the data processing unit 120 classifies some randomly selected data of the normal-state data and the abnormal-state data classified from the ECG data provided by the data collection unit 110 into the training dataset, and classifies the remaining data into a sample set for testing or validating the neural network model. Here, the sample set is classified into a validation set for validating the performance of the neural network model and a test set for testing the neural network model, and a ratio of the training dataset, the validation set, and the test adopts 6:2:2.

The model generation step S130 is a step in which the model generation unit 130 constructs a neural network model for predicting the hyperkalemia using the electrocardiogram based on the training dataset. Here, the neural network model adopts a convolutional neural network (CNN) model.

The validation step S140 is a step of analyzing the performance of the neural network model by applying the sample set to the neural network model. Here, the validation module 140 calculates the precision and recall of the corresponding neural network model, and calculates performance based on the calculated precision and recall by applying the validation set and the test set of the sample set to the neural network model. Meanwhile, the neural network model is constructed using the hyperkalemia prediction algorithm constructing system 100 through the electrocardiogram according to the present invention. Electrocardiogram data obtained from 855 patients who had symptoms of the hyperkalemia at least once at a local emergency center from July 2009 to June 2019 are used. Of these, 555 have chronic renal failure (CRF), and the rest are not diagnosed with the CRF. Information on the patients is shown in Table 2 below.

TABLE 2

| Datasets | | Non CRF | CRF | Total |
|---|---|---|---|---|
| Gender | Female | 496 | 747 | 1,243 |
| | Male | 672 | 1,043 | 1,715 |
| | total | 1,168 | 1,790 | 2,958 |
| Age | | 70.3 ± 19.0 | 72.6 ± 13.2 | 71.7 ± 15.8 |
| Height | | 155.5 ± 26.2 | 159.4 ± 14.4 | 158.0 ± 19.7 |
| Weight | | 58.8 ± 15.5 | 62.2 ± 12.2 | 60.9 ± 13.6 |

| | | | |
|---|---|---|---|
| Myocardial infarction | 35 | 117 | 152 |
| Heart failure | 116 | 271 | 387 |
| Angina | 93 | 235 | 328 |
| Diabetes | 251 | 912 | 1,163 |
| Hypertension | 323 | 1,037 | 1,360 |

Here, the data processing unit 120 processes three types of training datasets using the ECG data collected by the data collection unit 110. First, the data processing unit 120 classifies the ECG data at the time of the symptom onset of the hyperkalemia among the ECG data provided by the data collection unit 110 into the abnormal-state data, and classifies the ECG data from the time of the symptom onset of the hyperkalemia to the time before the first reference time into the normal-state data to generate a first dataset (dataset I), and set some of the first dataset (dataset I) as a first training dataset.

Further, the data processing unit 120 classifies the ECG data from the time of the symptom onset of the hyperkalemia to the time after the second reference time among the ECG data provided by the data collection unit 110 into the normal-state data, and classifies the ECG data at the time of the symptom onset of the hyperkalemia into the abnormal-state data to generate a second dataset (dataset II), and set some of the second dataset (dataset II) as a second training dataset.

In addition, the data processing unit 120 classifies, as the normal-state data, ECG data other than the ECG data among the ECG data from the time of the symptom onset of the hyperkalemia from the time before the first reference time from the time of the symptom onset of the hyperkalemia to the time after a second reference time from the time of the symptom onset of the hyperkalemia in the ECG data provided by the data collection unit 110, and classifies the ECG data at the time of the symptom onset of the hyperkalemia of among the ECG data provided by the data collection unit 110 into the abnormal-state data to generate a third dataset (dataset III). At this time, the data processing unit 120 sets some of the dataset (dataset III) as a third training dataset.

In Table 3 below, information on the first to third datasets (datasets I, II, and III) classified by the data processing unit 120 is disclosed.

TABLE 3

| Datasets | Dataset I | Dataset II | Dataset III |
|---|---|---|---|
| Training set | 1,186 | 879 | 1,426 |
| Validation set | 296 | 220 | 357 |
| Test set | 370 | 275 | 446 |
| Total | 1,852 | 1,374 | 2,229 |

Here, training set represents the training dataset, Validation set represents the validation set, and Test set represents the test set. Next, the model generator 130 generates the neural network model for each dataset. In addition, the validation module 140 calculates the performance of the neural network model constructed for each dataset using the validation set and the test set of each dataset.

Table 4 below shows an analysis result of the performance of the neural network model generated by the first dataset (dataset I).

TABLE 4

| Index | Events | Lead I | Lead II | V1 | V2 | V3 | V4 | V5 | V6 |
|---|---|---|---|---|---|---|---|---|---|
| Precision | Normal | 0.52 | 0.96 | 0.47 | 0.61 | 0.56 | 0.66 | 0.51 | 0.54 |
| | Hyperkalemia | 0.61 | 0.94 | 0.63 | 0.70 | 0.63 | 0.71 | 0.64 | 0.63 |
| Recall | Normal | 0.48 | 0.93 | 0.56 | 0.66 | 0.51 | 0.66 | 0.50 | 0.60 |
| | Hyperkalemia | 0.64 | 0.97 | 0.54 | 0.65 | 0.68 | 0.71 | 0.65 | 0.58 |
| F1- | Normal | 0.50 | 0.94 | 0.51 | 0.64 | 0.53 | 0.66 | 0.50 | 0.57 |
| score | Hyperkalemia | 0.62 | 0.95 | 0.58 | 0.68 | 0.66 | 0.71 | 0.65 | 0.60 |

Table 5 below shows an analysis result of the performance of the neural network model generated by the second dataset (dataset II).

TABLE 5

| Index | Events | Lead I | Lead II | V1 | V2 | V3 | V4 | V5 | V6 |
|---|---|---|---|---|---|---|---|---|---|
| Precision | Normal | 0.31 | 0.88 | 0.28 | 0.36 | 0.28 | 0.52 | 0.36 | 0.51 |
| | Hyperkalemia | 0.75 | 0.93 | 0.74 | 0.73 | 0.80 | 0.79 | 0.76 | 0.72 |
| Recall | Normal | 0.22 | 0.85 | 0.17 | 0.27 | 0.23 | 0.50 | 0.28 | 0.30 |
| | Hyperkalemia | 0.82 | 0.95 | 0.84 | 0.81 | 0.84 | 0.81 | 0.82 | 0.84 |
| F1- | Normal | 0.26 | 0.84 | 0.21 | 0.31 | 0.25 | 0.51 | 0.31 | 0.38 |
| score | Hyperkalemia | 0.78 | 0.94 | 0.79 | 0.77 | 0.82 | 0.80 | 0.79 | 0.79 |

Table 6 below shows an analysis result of the performance of the neural network model generated by the third dataset (dataset III).

TABLE 6

| Index | Events | Lead I | Lead II | V1 | V2 | V3 | V4 | V5 | V6 |
|---|---|---|---|---|---|---|---|---|---|
| Precision | Normal | 0.56 | 0.95 | 0.53 | 0.68 | 0.65 | 0.69 | 0.57 | 0.61 |
| | Hyperkalemia | 0.47 | 0.94 | 0.74 | 0.59 | 0.57 | 0.60 | 0.60 | 0.51 |
| Recall | Normal | 0.61 | 0.96 | 1.00 | 0.70 | 0.62 | 0.63 | 0.68 | 0.59 |
| | Hyperkalemia | 0.42 | 0.93 | 0.00 | 0.57 | 0.60 | 0.66 | 0.48 | 0.53 |
| F1- | Normal | 0.58 | 0.96 | 0.70 | 0.69 | 0.64 | 0.66 | 0.62 | 0.60 |
| score | Hyperkalemia | 0.44 | 0.94 | 0.00 | 0.58 | 0.59 | 0.63 | 0.53 | 0.52 |

Here, Precision represents the precision of the neural network model, Recall represents the recall of the neural network model, F1-score represents information on the performance of the neural network model, and ‘Lead I, Lead II, V1, V2, V3, V4, V5, V6’ represent electrode types from which the ECG data used to construct each neural network model is obtained. Referring to the table, the neural network model constructed based on electrocardiogram data of hyperkalemia patients provides relatively high performance. In particular, it can be seen that among the neural network models, the performance of the neural network model generated using the first dataset consisting of electrocardiogram data obtained from Lead II is the best.

According to the present invention, in the system 100 for constructing a hyperkalemia prediction algorithm through an electrocardiogram and the method for constructing the hyperkalemia prediction algorithm through the electrocardiogram, since the neural network model can be constructed for determining whether a patient has the hyperkalemia using the electrocardiogram, there is an advantage in being able to more easily diagnose patients with the hyperkalemia in a non-invasive manner using the neural network model.

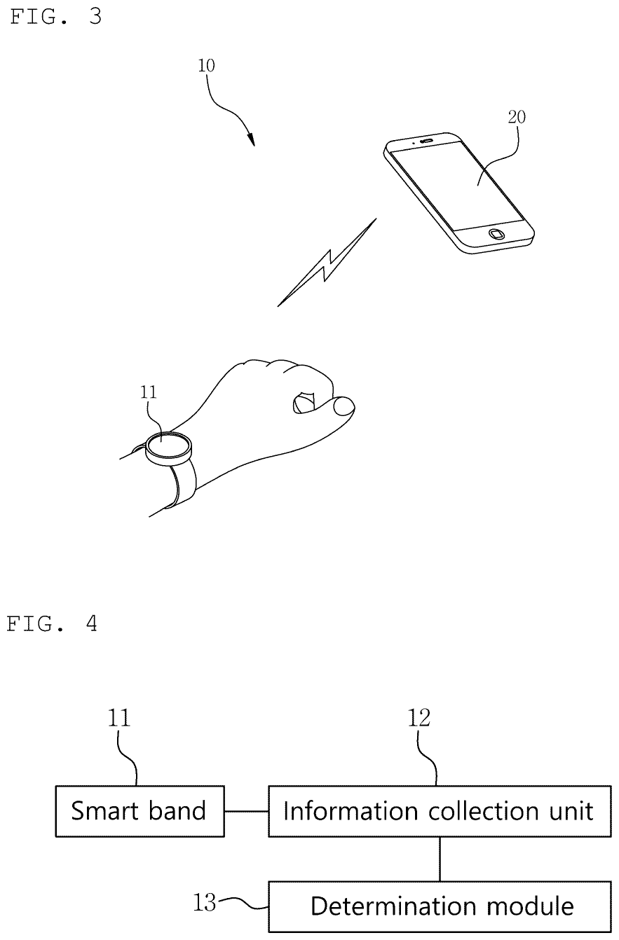

Meanwhile, FIGS. 3 and 4 illustrate a hyperkalemia prediction system 10 using the hyperkalemia prediction algorithm constructing system 100 through the electrocardiogram according to the present invention.

Referring to the drawing, the hyperkalemia prediction system 10 using the electrocardiogram includes a smart band 11 worn by a user, and measuring an electrocardiogram of the user, an information collection unit 12 collecting information on the electrocardiogram, a determination module 13 determining whether the user has thee hyperkalemia by applying the electrocardiogram of the user collected by the information collection unit 12 to a neural network model pre-constructed to predict the hyperkalemia according to the electrocardiogram, and a hyperkalemia prediction algorithm constructing system 100 through the electrocardiogram constructing the neural network mode using electrocardiogram data of the hyperkalemia patient, and providing the constructed neural network model to the determination module 13.

Although not illustrated in the drawing, the smart band 11 includes a wearing body that is enabled to be worn on the user's body, a memory installed on the wearing body and storing unique identification information and the user's personal information, a measurement sensor measuring the electrocardiogram of the user, and a communication module for transmitting the information stored in the memory and a measurement value of the measurement sensor.

The wearing body may be formed in the form of a wristwatch or wrist band to be worn on the user's wrist. Meanwhile, the wearing body is not limited thereto and may be formed in a form worn on the human body, but may also be formed in the form of an attachment sticker or pad that can be attached to the body.

The memory stores personal information such as the user's name, gender, age, etc., and identification information for identifying the smart band 11.

The communication module is for communication with a portable terminal 20, such as a user's smart phone, and Bluetooth Low Energy (BLE) for low-power operation is applied. Meanwhile, the communication module is not limited thereto and can adopt any communication means that can transmit data such as measurement values of the measurement sensor.

The measurement sensor is installed on the wearing body so as to be in contact with the user's body and measures an electrocardiogram signal of the user. Here, since the measurement sensor is an electrocardiogram measurement device commonly used in the prior art to measure the user's electrocardiogram, a detailed description will be omitted.

The information collection unit 12 is installed in a portable terminal 20 of the user and receives information on the user's electrocardiogram transmitted from the smart band 11 through the portable terminal 20, and delivers the received information on the electrocardiogram to the determination module 13. At this time, it is desirable for the information collection unit 12 to organize and store information in chronological order.

The determination module 13 determines whether the user has the hyperkalemia by applying the user's electrocardiogram collected by the information collection unit 12 to the neural network model. Here, the discrimination module 13 may display the determined information to the user through the user's portable terminal 20. At this time, the determination module 13 and the information collection unit 12 are preferably formed in the form of an application installed on the portable terminal 20.

The hyperkalemia prediction system 10 using the electrocardiogram according to the present invention, configured as described above, determines whether the patient has the hyperkalemia by applying the electrocardiogram measured through the smart band 11 worn by the user to the constructed neural network model, so the hyperkalemia prediction system 10 has an advantage of being able to more easily diagnose patients with the hyperkalemia.

The hyperkalemia prediction algorithm constructing system 100 through the electrocardiogram includes a data collection unit 110 collecting electrocardiogram data of multiple hyperkalemia patients, a data processing unit 120 generating a training data set for machine learning based on the electrocardiogram data collected by the data processing unit 120, and a model generation unit 130 constructing a neural network model for predicting a hyperkalemia using an electrocardiogram based on the training data set provided by the data processing unit 120. The hyperkalemia prediction algorithm constructing system 100 through the electrocardiogram is the same as the hyperkalemia prediction algorithm constructing system 100 through the electrocardiogram illustrated in FIG. 2, so a detailed description is omitted.

The description of the presented exemplary embodiments is provided so that those skilled in the art use or implement the present invention. Various modifications of the exemplary embodiments will be apparent to those skilled in the art and general principles defined herein can be applied to other exemplary embodiments without departing from the scope of the present invention. Therefore, the present invention is not limited to the exemplary embodiments presented herein, but should be analyzed within the widest range which is coherent with the principles and new features presented herein.

The description of the presented exemplary embodiments is provided so that those skilled in the art use or implement the present invention. Various modifications of the exemplary embodiments will be apparent to those skilled in the art and general principles defined herein can be applied to other exemplary embodiments without departing from the scope of the present invention. Therefore, the present invention is not limited to the exemplary embodiments presented herein, but should be analyzed within the widest range which is coherent with the principles and new features presented herein.

What is claimed is:

1. A system for constructing a hyperkalemia prediction algorithm through an electrocardiogram, the system comprising:
a data collection unit collecting electrocardiogram data of multiple hyperkalemia patients;
a data processing unit generating a training dataset for machine learning based on the electrocardiogram data collected by the data collection unit; and
a model generation unit constructing a neural network model for predicting a hyperkalemia using an electrocardiogram based on the training dataset provided by the data processing unit.

2. The system of claim 1, wherein the data collection unit collects electrocardiogram data of patients who have developed symptoms of hyperkalemia.

3. The system of claim 2, wherein the data collection unit collects electrocardiogram data of the hyperkalemia patient data from a time before a first preset reference time from the time of the symptom onset of the hyperkalemia to a time after a second preset reference time from the time of the symptom onset of the hyperkalemia.

4. The system of claim 2, wherein the data processing unit classifies the ECG data at the time of the symptom onset of the hyperkalemia among the ECG data provided by the data collection unit into abnormal-state data, and classifies the ECG data from the time of the symptom onset of the hyperkalemia to a time before a first preset reference time into normal-state data, and generates the training dataset to include the normal-state data and the abnormal-state data which are classified.

5. The system of claim 2, wherein the data processing unit classifies the ECG data at the time of the symptom onset of the hyperkalemia among the ECG data provided by the data collection unit into abnormal-state data, and classifies the ECG data from the time of the symptom onset of the hyperkalemia to a time after a second preset reference time into normal-state data, and generates the training dataset to include the normal-state data and the abnormal-state data which are classified.

6. The system of claim 2, wherein the data processing unit classifies the ECG data at the time of the symptom onset of the hyperkalemia among the ECG data provided by the data collection unit into the abnormal-state data, and also classifies, into the normal-state data, ECG data other than the ECG data among the ECG data from the time before the first preset reference time from the time of the symptom onset of the hyperkalemia to the time after the second preset reference time from the time of the symptom onset of the hyperkalemia, and generates the training dataset to include the normal-state data and the abnormal-state data which are classified.

7. The system of claim 4, wherein the data processing unit classifies some randomly selected data of the data classified from the ECG data provided by the data collection unit into the training dataset, and classifies the remaining data into a sample set for testing or validating the neural network model.

8. The system of claim 1, wherein the ECG data collected by the data collection unit is ECG lead II signals data.

9. The system of claim 2, wherein the symptom of the hyperkalemia is chronic renal failure (CRF).

10. The system of claim 7, further comprising:
a validation module analyzing the performance of the neural network model by applying the sample set to the neural network model.

11. A method for constructing a hyperkalemia prediction algorithm through an electrocardiogram, the method comprising:
a data collection step of colleting, by a data collection unit, electrocardiogram data of multiple hyperkalemia patients;
a data processing step of generating, by a data processing unit, a training dataset for machine learning based on the electrocardiogram data collected in the data collection step; and
a model generation step of constructing, by a model generation unit, a neural network model for predicting a hyperkalemia using an electrocardiogram based on the training dataset.

12. The method of claim 11, wherein in the data collection step, electrocardiogram data of patients who have developed symptoms of hyperkalemia are collected.

13. The method of claim 12, wherein in the data collection step, electrocardiogram data of the hyperkalemia patient data from a time before a first preset reference time from the time of the symptom onset of the hyperkalemia to a time after a second preset reference time from the time of the symptom onset of the hyperkalemia is collected.

14. The method of claim 12, wherein in the data processing step, the ECG data at the time of the symptom onset of the hyperkalemia among the ECG data collected in the data collection step is classified into abnormal-state data, and the ECG data from the time of the symptom onset of the hyperkalemia to a time before a first preset reference time is classified into normal-state data, and the training dataset is generated to include the normal-state data and the abnormal-state data which are classified.

15. The method of claim 12, wherein in the data processing step, the ECG data at the time of the symptom onset of the hyperkalemia among the ECG data collected in the data collection step is classified into abnormal-state data, and the ECG data from the time of the symptom onset of the hyperkalemia to a time after a second preset reference time is classified into normal-state data, and the training dataset is generated to include the normal-state data and the abnormal-state data which are classified.

16. The method of claim 12, wherein in the data processing step, the ECG data at the time of the symptom onset of the hyperkalemia among the ECG data collected in the data collection step is classified into the abnormal-state data, and ECG data other than the ECG data among the ECG data from the time before the first preset reference time from the time of the symptom onset of the hyperkalemia to the time after the second preset reference time from the time of the symptom onset of the hyperkalemia is classified into the normal-state data, and the training dataset is generated to include the normal-state data and the abnormal-state data which are classified.

17. The method of claim 14, wherein in the data processing step, some randomly selected data of the data classified from the ECG data collected in the data collection step are classified into the training dataset, and the remaining data are classified into a sample set for testing or validating the neural network model.

18. The method of claim 17, further comprising:
a validation step of analyzing the performance of the neural network model by applying the sample set to the neural network model.

19. A hyperkalemia prediction system using an electrocardiogram, comprising:
a smart band worn by a user, and measuring an electrocardiogram of the user;
an information collection unit collecting information on the electrocardiogram of the user measured by the smart band; and
a determination module determining whether the user has thee hyperkalemia by applying the electrocardiogram of the user collected by the information collection unit to a neural network model pre-constructed to predict the hyperkalemia according to the electrocardiogram.

20. The hyperkalemia prediction system using an electrocardiogram of claim 19, wherein the smart band is worn on a wrist of the user.

21. The hyperkalemia prediction system using an electrocardiogram of claim 19, further comprising:
a model construction unit constructing the neural network mode using electrocardiogram data of the hyperkalemia patient, and providing the constructed neural network model to the determination module.

22. The hyperkalemia prediction system using an electrocardiogram of claim 21, wherein the model construction unit includes
a data collection unit collecting electrocardiogram data of multiple hyperkalemia patients;
a data processing unit generating a training dataset for machine learning based on the electrocardiogram data collected by the data processing unit; and
a model generation unit constructing a neural network model for predicting a hyperkalemia using an electrocardiogram based on the training dataset provided by the data processing unit.

23. The hyperkalemia prediction system using an electrocardiogram of claim 22, wherein the data collection unit collects electrocardiogram data of the hyperkalemia patient data from a time before a first preset reference time from the time of the symptom onset of the hyperkalemia to a time after a second preset reference time from the time of the symptom onset of the hyperkalemia.

24. The hyperkalemia prediction system using an electrocardiogram of claim 22, wherein the data processing unit classifies the ECG data at the time of the symptom onset of the hyperkalemia among the ECG data provided by the data collection unit into abnormal-state data, and classifies the ECG data from the time of the symptom onset of the hyperkalemia to the time before the first preset reference time into normal-state data, and generates the training dataset to include the normal-state data and the abnormal-state data which are classified.

25. The hyperkalemia prediction system using an electrocardiogram of claim 22, wherein the data processing unit classifies the ECG data at the time of the symptom onset of the hyperkalemia among the ECG data provided by the data collection unit into abnormal-state data, and classifies the ECG data from the time of the symptom onset of the hyperkalemia to the time after the second preset reference time into normal-state data, and generates the training dataset to include the normal-state data and the abnormal-state data which are classified.

26. The hyperkalemia prediction system using an electrocardiogram of claim 22, wherein the data processing unit classifies the ECG data at the time of the symptom onset of the hyperkalemia among the ECG data provided by the data collection unit into the abnormal-state data, and classifies, into the normal-state data, ECG data other than the ECG data among the ECG data from the time before the first preset reference time from the time of the symptom onset of the hyperkalemia to the time after the second preset reference time from the time of the symptom onset of the hyperkalemia, and generates the training dataset to include the normal-state data and the abnormal-state data which are classified.

27. The hyperkalemia prediction system using an electrocardiogram of claim 24, wherein the data processing unit classifies some randomly selected data of the data classified from the ECG data provided by the data collection unit into the training dataset, and classifies the remaining data into a sample set for testing or validating the neural network model.

28. The hyperkalemia prediction system using an electrocardiogram of claim 27, further comprising:
a validation module analyzing the performance of the neural network model by applying the sample set to the neural network model.
